(19) United States
(12) Patent Application Publication
(10) Pub. No.: US 2006/0048287 A1
(43) Pub. Date: Mar. 9, 2006

(54) Frameless diving face mask

(76) Inventor: Tzong-Fuh Kuo, Hsin Juang City (TW)

Correspondence Address: Keith Kline;PRO-TECHTOR INTERNATIONAL SERVICES, 20775 Norada Court, Saratoga, CA, 95070-3018, US

(21) Appl. No.: 10/923,903

(22) Filed: Aug. 21, 2004

(51) Int. Cl. A61F 9/02

(52) U.S. Cl. 2/426

(57) ABSTRACT

A frameless diving face mask embodying a plastic fabricated hard headgear fixedly clasp to the frameless diving face mask. Wherein the headgear is not only color changeable, moreover, can be fabricated to display varied trademarks, brand logos, and so on. Furthermore, a lug and a soft elastic member are configured on an outer edge of each of two sides of a lens frame. Upon a lug configured on each side of the lens frame being disposedly clipped between two locating blocks, thereby enabling side fasteners to clasp onto the lugs and form an integral body with the lens frame thereof, rupture and leakage of the lens frame is prevented, and, moreover, restoring elasticity of the elastic members effectuate rapid restoring of the side fasteners to their original positions of compactness with the lens frame.

### BACKGROUND OF THE INVENTION

(a) Field of the Invention

The present invention relates to a diving face mask, and more particularly to the diving face mask having a color changeable headgear fixedly clasped atop a lens frame of the diving face mask. Upon a lug and a soft elastic member configured on an outer edge of each of two sides of the lens frame being connected to side fasteners, apart from preventing rupture and leakage of the lens frame, moreover, restoring elasticity of the elastic members effectuate rapid restoring of the side fasteners to their original positions of compactness with the lens frame.

(b) Description of the Prior Art

A conventional frameless diving face mask, as depicted in FIG. 1, is structured to comprise a lens frame 100 fabricated from soft silica gel and transparent glass lenses 200. Because of difficulty in combining the soft silica gel lens frame 100 with other component members, the lens frame 100 is generally black or of a single transparent color, and as a result lacks eye appeal. Furthermore, a side fastener 300 is separately configured on each of two sides of the lens frame 100, and a buckle 401 configured on each of two ends of a head strap 400 is inserted and thereby fastened interior of the side fasteners 300, forming a single body thereof. A short insert pin 301 is utilized to pass through each of the side fasteners 300 and a flange 101 on each side of the lens frame 100, thus fixedly attaching the side fasteners 300 onto the flanges 101, which thereby allows the side fasteners 300 to easily sway about the insert pins 301.

Referring to FIGS. 2 and 3, which show another diving face mask configured with side fasteners 500, each having length greater than width of a lens 200, and wherein each side of the diving face mask utilizes a long insert pin 501 to penetrate top and bottom through holes 502 of each of the side fasteners 500, and, moreover, passes through a vertical long slot 102 of the lens frame 100, thereby securing the side fasteners 500, and preventing same from swaying. However, the long slot 102 of the lens frame 100 proximates an inner edge of the lens frame 100. However, after the diving face mask has been immersed in seawater over a long period of time, rupture and leaking of the lens frame 100 easily results.

### SUMMARY OF THE INVENTION

A primary objective of the present invention is to fixedly clasp a headgear to a soft silica gel lens frame of a conventional diving face mask. Wherein the headgear is not only color changeable, moreover, can be fabricated to display varied trademarks, brand logos, and so on. Furthermore, a lug and a soft elastic member are configured on an outer edge of each of two sides of the lens frame, wherein the elastic members provide restoring elasticity that effectuates restoring side fasteners to their original positions, and the lugs provide for the side fasteners to connect therewith, and which thereby effectuates preventing rupture and leakage of the lens frame.

### BRIEF DESCRIPTION OF THE DRAWINGS

FIG. 1 shows a general view of a conventional diving face mask.

FIG. 2 shows general view of another conventional diving face mask.

FIG. 3 shows an exploded elevational schematic view of the conventional diving face mask.

FIG. 4 shows a general view according to the present invention.

FIG. 5 shows an exploded elevational schematic view according to the present invention.

FIG. 6 shows a partial side view according to the present invention.

FIG. 7 shows an assembled elevational schematic view of an embodiment according to the present invention.

FIG. 8 shows a partial side view of the embodiment according to the present invention.

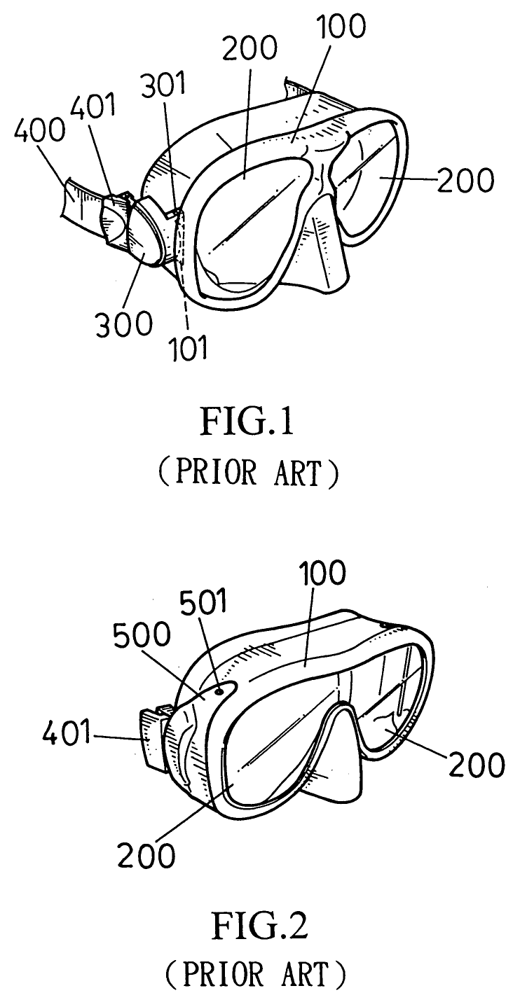

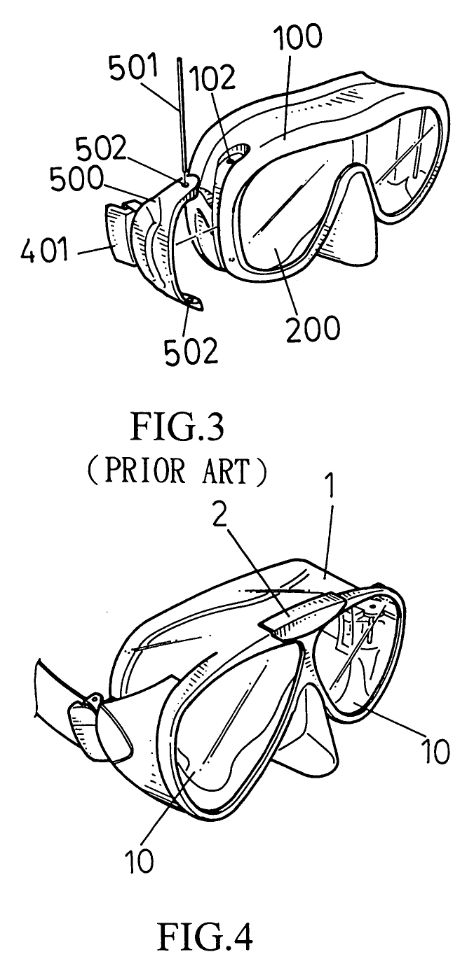

### DETAILED DESCRIPTION OF THE PREFERRED EMBODIMENTS

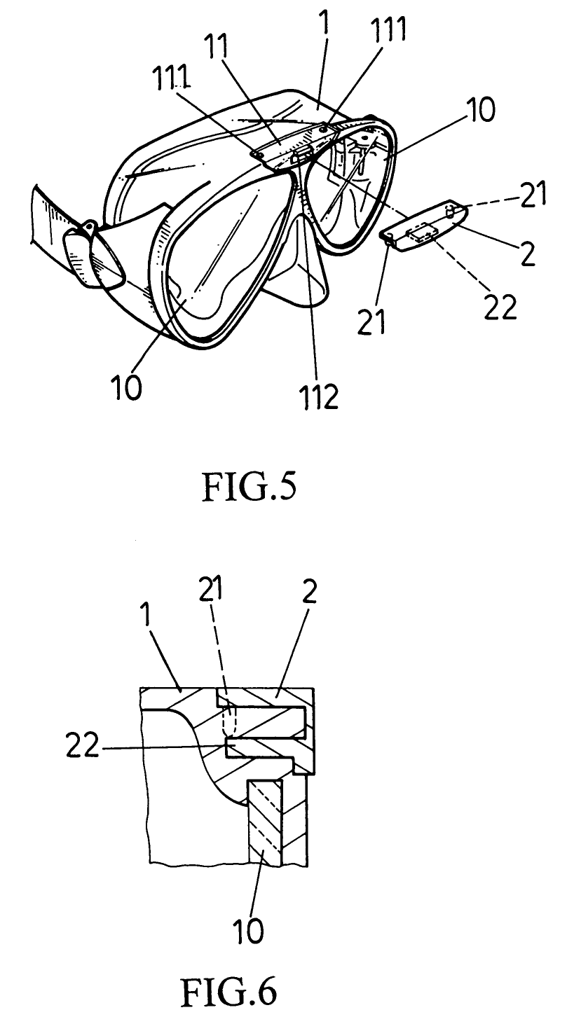

Referring to FIGS. 4, 5 and 6, which show the present invention structured to comprise a lens frame 1 and glass lenses 10, wherein a L-shaped groove 11 is defined central of a top portion of the soft silica gel lens frame 1. A locating hole 111 is defined on each of two sides of the groove 11, and a clasp slot 112 is provided beneath the groove 11. A headgear 2 can be clasped into the groove 11, and from a side view the headgear 2 assumes an inverted L-shape. A locating pin 21 is configured on each of two sides of a bottom portion of the headgear 2. The locating pins 21 can separately clasp interior of the locating holes 111 of the lens frame 1. An inner side of a front edge of the headgear 2 is provided with a clasp 22, which can insert into the clasp slot 112 of the lens frame 1, and thereby fixedly position the headgear 2 therein.

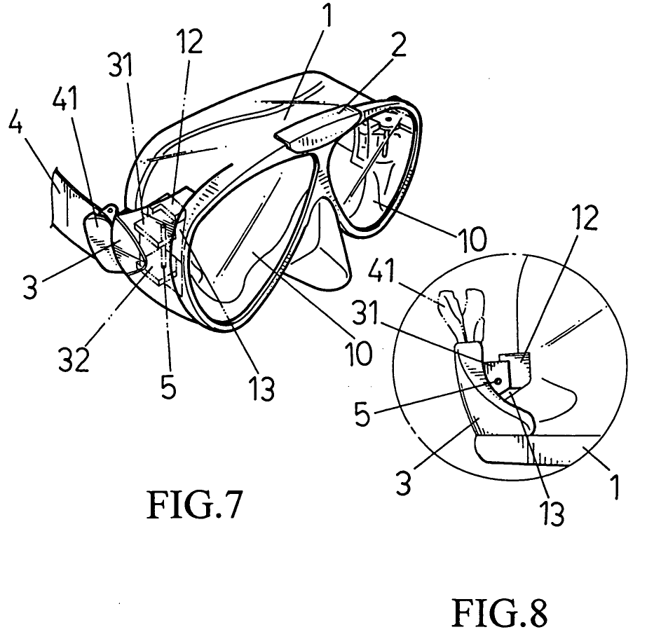

Referring to FIGS. 7 and 8, which show the present invention structured to comprise the lens frame 1, the lenses 10 and two side fasteners 3. The lenses 10 are embedded interior of the lens frame 1, and the two side fasteners 3 are separately firmly configured to each of two sides of the lens frame 1. Interior of each of the side fasteners 3 is pin connected to a buckle 41 of a head strap 4. The present invention is characterized in that:

An outwardly protruding triangular lug 12 and an inverted L-shaped soft elastic member 13 are configured on each of the two sides of the lens frame 1. An upper and lower end of an inner side of the side fastener 3 is provided with locating blocks 31 and 32. The lug 12 of the lens frame 1 is disposedly configured between the two locating blocks 31 and 32, thereby allowing the two locating blocks 31 and 32 to clip round outer sides of the lug 12. A locating pin 5 passes through the locating block 31, the lug 12 and the lower locating block 32 in sequence on each of the two sides of the lens frame 1, and which thereby enables the side fasteners 3 to clasp onto the lugs 12. Because the two locating blocks 31 and 32 are closely connected to the elastic member 13, upon the head strap 4 pulling on the side fasteners 3, restoring to original position of compactness with the lens frame 1 is rapidly effectuated by means of restoring elasticity of the elastic member 13. In addition, each of the lugs 12 of the lens frame 12 is distanced from an outer side of the lens frame 1, thus, integral connection of the two locating blocks 31 and 32 with the side fastener 3 by means of the locating pin 5 eliminates rupture and leakage of the lens frame 1.

In conclusion, the present invention fixedly clasps a protruding headgear to a main body of a top end of a conventional diving face mask. Furthermore, the outwardly protruding elastic member 13 and the lug 12 are configured on each of the two sides of the lens frame 1, and integral connection of the two locating blocks 31 and 32 with the side fastener 3 by means of the locating pin 5 not only allows prevention of rupture and leakage of the lens frame 1, moreover, restoring elasticity of the elastic members 13 effectuate rapid restoring of the side fasteners 3 to their original positions of compactness with the lens frame 1.

What is claimed is:

1. A frameless diving face mask comprising a lens frame, lenses, and two side fasteners, the lenses are embedded interior of the lens frame, and the two side fasteners are separately firmly configured to each of two sides of the lens frame; and characterized in that:
a groove is defined in the lens frame, and a headgear is clasped into the groove; a locating pin is utilized to clasp interior of a locating hole defined in the groove of the lens frame, thereby forming an integral body with the lens frame; a lug and an elastic member are configured on each of the two sides of the lens frame, an inner edge of each of the side fasteners is provided with locating blocks, and each of the side fasteners is connected to the lug and the locating blocks by means of a locating pin, thereby enabling the side fasteners and the lens frame to form an integral body therefrom; upon the side fasteners being pulled by a head strap, restoring elasticity of the elastic members rapidly effectuate restoring of the side fasteners to original positions of compactness with the lens frame.

2. The frameless diving face mask according to claim 1, wherein a clasp slot is provided beneath the groove of the lens frame, an inner side of a front edge of the headgear is provided with a clasp, which can insert into the clasp slot of lens frame, and thereby fixedly position the headgear therein.

3. The frameless diving face mask according to claim 1, wherein the elastic member of the lens frame is soft and of an inverted L-shaped form.

4. The frameless diving face mask according to claim 1, wherein each of the lugs of the lens frame is of a triangular shaped from.
